(19) United States
(12) Patent Application Publication
(10) Pub. No.: US 2015/0216713 A1
(43) Pub. Date: Aug. 6, 2015

(54) Protective soft restraint system to secure both adults and children

(71) Applicant: Prime Time Products, LLC, Phoenix, AZ (US)

(72) Inventor: James Arthur Mabry, Phoenix, AZ (US)

(73) Assignee: Prime Time Products, LLC, Phoenix, AZ (US)

(21) Appl. No.: 14/171,453

(22) Filed: Feb. 3, 2014

(51) Int. Cl. A61F 5/37

(52) U.S. Cl. CPC A61F 5/3769

(57) ABSTRACT

This invention is a universal, cost effective, disposable, restraint strap system. This invention provides a way for emergency medicine field providers to quickly and effectively secure their patients to a rigid type backboard to immobilize the patient. This immobilization will facilitate safe and secure movement of the patient to and from transport vehicles. The securing straps on this invention easily adjust allowing best positioning of the straps depending on the circumstances presented. Specifically, the straps adjust allowing the user to position the straps for optimal placement depending on the size of the patient (both adult and child).

### CROSS-REFERENCE TO RELATED APPLICATIONS

This application claims the priority of Provisional Application No. 61/850,760, filed on Feb. 22, 2013.

### STATEMENT REGARDING FEDERALLY SPONSORED RESEARCH OR DEVELOPMENT

Not Applicable

### FIELD OF INVENTION

This invention relates to a medical device used during medical emergencies. Specifically, a medical restraint strap used to secure a patient to a rigid backboard.

### BACKGROUND

Various devices have been created and used to immobilize a patient's body while transporting the patient from an accident site to a treatment site such as a hospital emergency room. In the case of an automobile accident, the patient must be transported from the injury scene to a medical facility. This type of scenario employs adverse conditions involving time, stress, weather, and/or darkness. To best transport the patient, to minimize possible aggravation of injuries to a patient's cervical spine, head and neck, medical personnel must quickly and efficiently secure the patient on a rigid board, often referred to as a backboard, spine board, or trauma board.

While various devices are available for securing and restraining a patient's body to a backboard during patient transport, problems with these systems prevail. It has been found with some patient restraint systems, the straps need to be cleaned and sterilized after every use. This creates cost to sterilize, and possible spread of the biohazards and contaminants (blood or other fluids) which have come into contact with the straps.

Additionally, other types of straps have to be moved off of the board so that the patient can be placed on the board without any hold-down straps resting under the patient. It can be appreciated that if a strap is inadvertently located on a board beneath the patient, the patient has to be moved to recover the strap. Other devices might require part of the device be placed under part or all of the patient. When a strap requires specific placement to properly secure the patient, it can easily cause the strap to be misplaced. This problem of strap misplacement is particularly prevalent at a night accident scene where there is low visibility, a need for quick actions, or heightened stress.

Similarly, some strap systems are specialized such that additional straps, or different styled straps, are located on one end of the strap system. These additional or different straps would be used to assist in immobilizing the head. If these straps are placed backwards, the patient needs to be rotated to accommodate the straps, or the straps would need to be repositioned. Also, these types of straps can cause unwanted confusion, increase time, or improper use during the adverse conditions of a medical emergency.

Further, many straps work well for adult patients, but the location of the straps and buckles make them ineffective for use with shorter patients, such as children. Although some straps have been created for use with children, those straps are separate from the straps used for adults.

What is desired is a strap system to properly secure a patient to a backboard which has the ability to be adjustable to the size of the patient. A strap that is fast, easy to use, and effective.

### SUMMARY OF THE INVENTION

The present invention relates to a system of restraint straps to be used in the emergency medical field. Specifically, the restraint straps are to be used in securing a patient to a rigid type backboard to immobilize the patient. When the patient is effectively immobilized and secured to a rigid backboard, the patient is better protected from further injury during transportation of the patient to a treatment facility.

The restraint straps (hereafter referred to as the “invention”) will give a way for emergency personnel to quickly and effectively secure any sized patient to a rigid backboard.

This invention is removed from its packaging and placed on top of the patient already lying on the backboard. This invention does not need to be placed under the patient or require any further movement of the patient after the patient is placed on the backboard.

This invention also has symmetry which aids in the speed and ease of use. This invention is symmetrical, such that the straps cannot be placed upside down, or in the wrong direction. When the strap is unrolled and placed on top of the patient, the specialized straps used to immobilize the shoulders and neck are on both ends of the strap. This symmetry gives the user ease and speed while performing their tasks.

Another great aspect of this invention is that the user can adjust the length of the mid-line strap. On the bottom portion of the mid-line strap, it has loop and hook (Velcro or similar) on the upper and lower sides of the strap. The loop and hook on the upper and lower sides of the strap, allow the mid-line strap to be folded in two locations. Essentially, the mid-line strap is folded back on top of itself shortening the length of the mid-line strap. This shorter mid-line strap is ideal for use with a shorter patient such as a child.

In the case when the invention is to be used for a taller adult, the user would pull on the mid-line strap, unfolding the loop and hook length adjustment, making the mid-line strap longer. The securing straps, which wrap around the patient for immobilization, can slide along the mid-line strap to the necessary locations on the taller adult. The same strap system that could be used for a child, is also ideal for use with an adult.

The ability to move the secure straps along the mid-line strap has other benefits. The movement of the secure straps will allow the user to slightly adjust the location of the secure straps (on either child or adult) to avoid putting pressure on an unwanted area of the patient (such as a wound).

The invention is made from a disposable material. This invention is a one time use, therefore, eliminating any need to clean and sterilize the straps. This invention gives the user, the emergency personnel, a fast, easy, and accurate strap system to properly and effectively secure a patient to a backboard.

### BRIEF DESCRIPTION OF THE DRAWINGS

FIG. 1 is a top view of the invention, with all straps unrolled and laid flat, illustrating the embodiment of the invention.

FIG. 2 illustrates the embodiment of the adjustable means, and is a close up view of the mid-line strap's length adjustment. The mid-line strap's length adjustment is partially folded.

FIG. 3 is a close up view of the mid-line strap's length adjustment, fully extended, and laid flat.

FIG. 4 illustrates the embodiment of the loop means and is a close up view of the loop means.

### DETAILED DESCRIPTION OF THE DRAWINGS

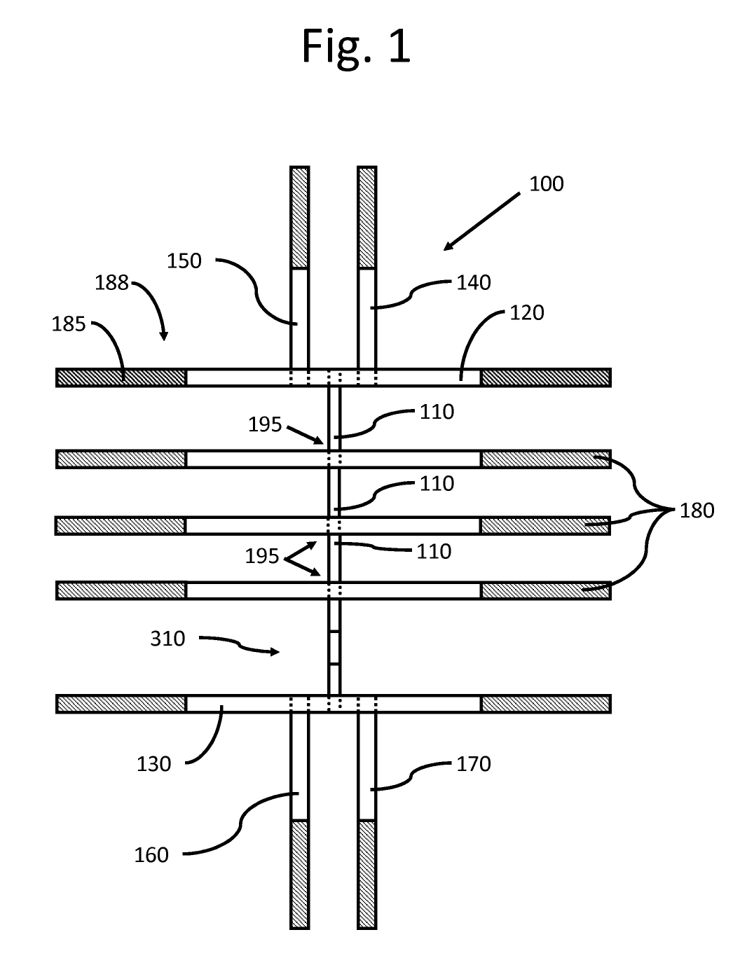

FIG. 1 shows the embodiment of the invention. The restraint strap system 100 can comprise a mid-line strap 110 which runs along the longitudinal axis. Attached at one end of the mid-line strap 110 is a top strap 120. The top strap 120 is oriented along the latitudinal axis. The top strap 120 is attached to the mid-line strap 110 near the center of the top strap 120.

Attached to the top strap 120 are two end straps 140, 150. The end straps 140, 150 are orientated on the longitudinal axis. One termination of these end straps 140, 150 is attached to the top strap 120. The first end strap 150 is placed just to the left of the mid-line strap 110, and the second end strap 140 is placed just to the right of the mid-line strap 110.

Attached to the bottom of the mid-line strap 110 is a bottom strap 130. The bottom strap 130 is orientated along the latitudinal axis. The bottom strap 130 is attached to the mid-line strap 110 near the center of the bottom strap 130. Attached to the bottom strap 130, are two end straps 160, 170. These end straps 160, 170 are orientated on the longitudinal axis. One end of these end straps 160, 170 is attached to the bottom strap 130. The third end strap 160 is placed just to the left of the mid-line strap 110, and the fourth end strap 170 is placed just to the right of the mid-line strap 110.

There are a number of body straps 180. The body straps 180 are located on the latitudinal axis. The body straps 180 have a loop means 195 in the center of the body strap 180. The loops meas 195 is to allow the mid-line strap 110 to pass through the body strap 180. The loop means 195 allows the body strap 180 to slide along the mid-line strap 110, sliding along the longitudinal axis. However, the loop means 195 keeps the body strap from moving along the latitudinal axis within the restraint strap system 100.

The ability to adjust the body straps 180 along the mid-line strap 110 gives the user the ability to wrap the body straps 180 around the patient in the best manner within the given circumstances.

Each strap included in the restraint system 100, except the mid-line strap 110, can comprise a attachment means 185. The attachment means 185 is illustrated by the shaded end of each strap about one fourth the length of each strap. The attachment means 188 can be comprised of a loop and hook system (Velcro or similar) 188. The shaded area 185 of the attachment means 188, represents the hook side and the white area 186 would represent the loop side of the loop and hook system 188. This attachment means 188 would work by allowing the user to pull any strap around an object or loop (ie: the handle holes on the backboard) and fold it back on top of the same strap and thereby securing the attachment means 185.

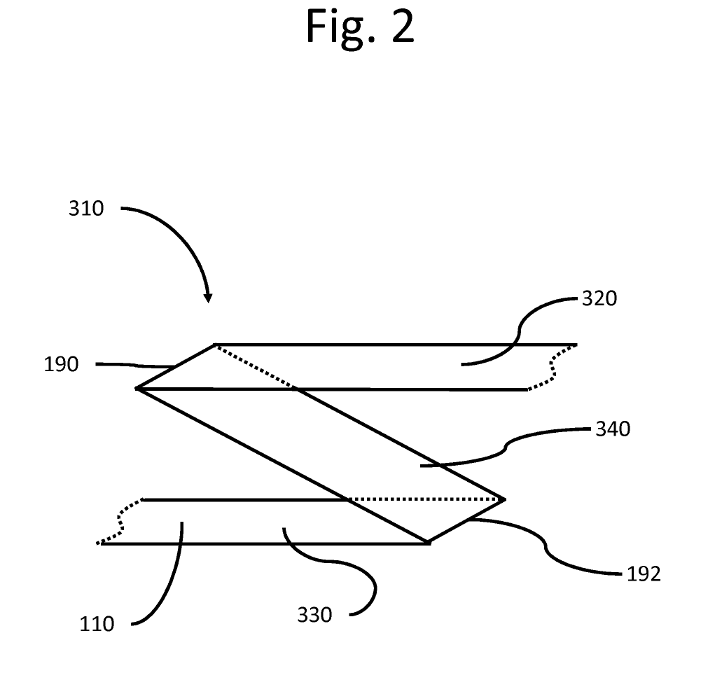

FIG. 2 illustrates the embodiment an adjustment means 310 of the restraint system 100. The mid-line strap comprises the adjustment means 310. The adjustment means 310 can be created by folding the mid-line strap on top of itself. This is done by creating two folds 190, 192. The first fold 190, folds the mid-line strap 110 back on top of the mid-line strap 110. The second fold 192 folds the mid-line strap 110 back the same direction the mid-line strap 110 was before the first fold 190. The two folds 190, 192 create three sections 320, 330, 340. These two folds 190, 192 create the adjustment means 310 to shorten or lengthen the mid-line strap 110, while still keeping the same features and uses of the restraint system 100.

A way to keep the adjustment means 310 secure while in the shortened length position, would be to add a loop and hook material (Velcro or similar) on the appropriate sides of the sections 320, 330, 340 created by the folds 190, 192. Section 320 would have loop material on bottom surface. Section 330 would have hook type material on the top surface as well as the bottom surface. Section 340 would have loop material on the top surface. Using the loop and hook material on the appropriate three sections 320, 330, 340, would keep the adjustment means 310 in the shortened length and would not inadvertently unfold. However, the loop and hook material would allow a user to pull on the ends of the mid-line strap 110, releasing the loop and hook bond, and thereby unfolding the adjustment means 310, making the restraint system 100 longer for use with a taller patient.

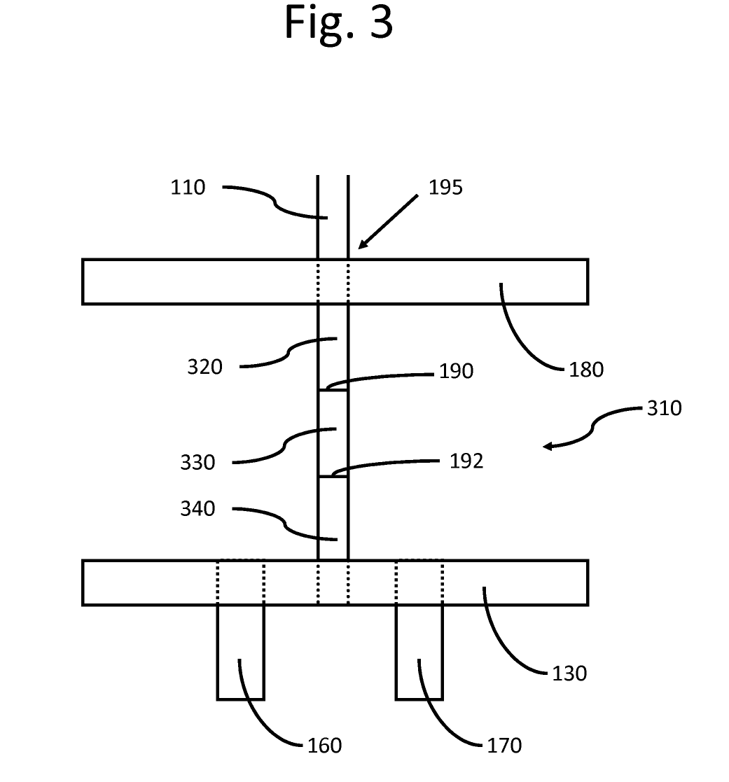

FIG. 3 illustrates the adjustment means 310. FIG. 3 is a closer view showing the same elements of the adjustment means 310 within the restraint system 100 as explained in FIG. 2. FIG. 3 illustrates the adjustment means 310 and the location of the adjustment means 310 within the restraint system 100. The adjustment means 310 is located above the bottom strap 130. Although FIG. 3 illustrates a body strap 180 above the adjustment means 310, the body strap 180 can move down the mid-line strap 110 by way of the loop means 195. The body strap 180 can slide over the adjustment means 310, when the adjustment means 310 is folded in the shortened length position, (as illustrated in FIG. 2). The body strap 180 can also slide over the adjustment means 310 in the extended, longer length position (as illustrated in FIG. 3).

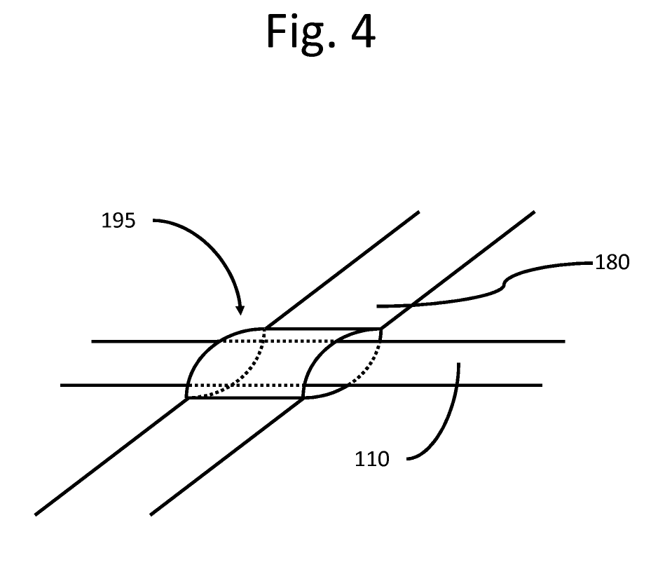

FIG. 4 illustrates a close view of the loop means 195. The body straps 180 each have the loop means 195 substantially near the center. The loop means 195 can be made of the same material used in the body strap 180.

The present invention is not limited to the above-described embodiments. It is apparent to one who has an ordinary skill in the art that there may be many modifications and variations within the same technical spirit of the invention.

What claimed is:

1. A restraint system of soft, flexible straps comprising:
a. a mid-line strap, having a top end and a bottom end, located on a longitudinal axis of the restraint system;
b. a top strap, on a lateral axis of the restraint system, which is attached to the top end of the mid-line strap near the center of the top strap,
c. a bottom strap, on the lateral axis of the restraint system, which is attached to the bottom end of the mid-line strap near the middle of the bottom strap,
d. a plurality of body straps, substantially on the lateral axis of the system, the plurality of body straps being located between the top strap and the bottom strap of the system;
e. each body strap included in the plurality of body straps having a loop means primarily in the middle of the body strap, the loop means allowing the mid-line strap to pass inside the loop means, the loop means giving the body strap the ability to move up and down along the complete longitudinal axis of the mid-line strap;
f. the mid-line strap having one or more adjustable means to adjust the length of the mid-line strap;
g. the top strap, the bottom strap, and each body strap, within the plurality of body straps, each having an affixing means.

2. The restraint system of claim 1, wherein the affixing means is a loop and hook (Velcro) type of material.

3. The restraint system of claim 2, wherein the hook type of material covers one fourth of the length of the strap from the end of each secure strap, and the loop type material covers the center half of each secure strap.

4. The restraint system of claim 1, wherein the system includes a plurality of end straps placed on the longitudinal axis of the system, one termination of each end strap is attached to the system.

5. The restraint system of claim 4, wherein the plurality of end straps include: a first end strap attached to the top strap, placed to the left of the mid-line strap, a second end strap attached to the top strap, placed to the right of the mid-line strap, a third end strap attached to the bottom strap, located to the left of the mid-line strap, a fourth end strap placed to the right of the mid-line strap;

6. The restraint system of claim 4, wherein the plurality of end straps each have an affixing means.

7. The restraint system of claim 6, wherein the affixing means is comprised of a loop and hook system, clasp, clip, or adhesive.

8. The restraint system of claim 5, wherein the first end strap, the second end strap, the third end strap, and the fourth end strap, are attached to the system at an angle to the longitudinal axis.

9. The restraint system of claim 1, wherein the loop means is made of the same material as the body straps.

10. The restraint system of claim 1, wherein the loop means is made of a different material, but still allowing the body strap to move along the longitudinal axis of the mid-line strap.

11. The restraint system of claim 1, wherein the loop means allows the body straps to pass over the one or more adjustment means of the mid-line strap.

12. The restraint system of claim 1, wherein the adjustment means includes a means to secure the mid-line strap in the shortened length position.

13. The restraint system of claim 12, wherein the means to secure the mid-line strap in the shortened length position, is created with a loop and hook type of material.

14. The restraint system of claim 12, wherein the means to secure the mid-line strap in the shortened length position is created with an adhesive, clasp, or clip.

15. The restraint system of claim 1, wherein the affixing means is a clasp, clip, or adhesive.

16. The restraint system of claim 1, wherein the plurality of body straps are at an angle to the lateral axis of the system.

17. The restraint system of claim 1, wherein all of the straps are arranged so that the system is substantially a mirror image along the longitudinal center line of the system.

18. The restraint system of claim 1, wherein all of the straps are arranged so that the system is substantially a mirror image along the latitude center line of the system.

19. The restraint system of claim 1, wherein the adjustable means on the mid-line strap is created by a loop and hook type material, clip, clasp, or an adhesive, and folding the mid-line strap over itself once, then folded back over itself again, to shorten the length of the mid-line strap.
